(19) United States
(12) Patent Application Publication
(10) Pub. No.: US 2006/0051861 A1
(43) Pub. Date: Mar. 9, 2006

(54) Cell lines for use in increasing protein yield from a cell culture

(75) Inventor: Thomas Primiano, Chicago, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Clonex Development, Inc., Chicago, IL (US)

(21) Appl. No.: 11/011,299

(22) Filed: Dec. 14, 2004

Related U.S. Application Data
(60) Provisional application No. 60/608,059, filed on Sep. 7, 2004.

(51) Int. Cl. C12P 21/06; C12N 5/06; C07K 14/72

(52) U.S. Cl. 435/325; 435/455; 435/69.1; 530/350

(57) ABSTRACT

The present invention provides cell lines useful in the production proteins and peptides. The cell lines contain recombinant expression constructs. The recombinant expression construct encodes the STPs consisting of the Cy protein motif and/or an ankyrin-binding protein motif. Each recombinant expression construct also contains an inducible transcription regulation element having for conditional expression of the senescence-triggering factors (STPs).

This application is related to U.S. Ser. No. 60/528,929, filed Dec. 11, 2003, and U.S. Ser. No. 60/608,059, filed Sep. 7, 2004, the disclosures of which are expressly incorporated by reference herein.

### BACKGROUND OF THE INVENTION

1. Technical Field

The invention relates to cell lines useful in the production of cellular proteins of commercial interest, particularly antibodies. The cell lines of the invention are engineered to contain a transcriptional regulatory system controlling the expression of senescence-triggering peptides (STPs) and may be used as host cell lines for producing recombinant proteins including therapeutic proteins, or as fusion partners, inter alia, for the production of monoclonal antibody-producing hybridomas from antigen-stimulated lymphocyte cells.

2. Background of the Related Art

Recombinant Protein Production

Genetic engineering and recombinant DNA techniques provide the capacity to produce desired proteins in cultured cells, thereby permitting production of proteins not previously available in useful quantities. Erythropoietin, tissue plasminogen activator, insulin and particularly antibodies of defined specificity (monoclonal antibodies) can be obtained by protein production in cultured cells heterologous to those cells that natively make the protein.

However, the capacity for a cultured cell to produce a heterologous protein must be matched with production of sufficient quantities of the protein to be useful. Hitherto, the art recognized only limited methods for increasing heterologous protein production outside screening for the serendipitous high producer; these methods, including cell type-specific promoters, enhancers and other regulatory sequences acting genetically in cis, and combinations of certain cis-acting factors with their cognate trans-acting regulatory activators, retained a high degree of unpredictability in achieving useful production levels of heterologous proteins. This was particularly a problem with hybridoma cell lines producing monoclonal antibodies, since the advantages of said cells in secreting monoclonal antibodies into the cell culture medium was offset by the difficulties and unpredictability of identifying a particular cellular clone that had sufficiently high antibody production levels to be useful.

In addition, the conditions of in vitro cell culture made it advantageous to permit the cell culture to grow to an appropriate number of cells prior to (preferably) decreasing cell proliferation and dramatically increasing heterologous protein production. In this way, the maximum number of cells would be capable of each producing high levels of the heterologous protein, thereby maximizing the overall yield of the culture. Although some success in perfecting these methods was known in the art using prokaryotic cells, the methods had not been applied with equal success when using eukaryotic cells (which generally are more appropriate for heterologous protein production due to, inter alia, eukaryote-specific post-translational modifications, secretion and other desirable properties.

There is thus a need in the art to identify methods and reagents for increasing heterologous protein production in cells, particularly eukaryotic cells including hybridoma cells that make monoclonal antibodies.

### SUMMARY OF THE INVENTION

This invention provides reagents and methods for increasing heterologous protein production in cultured eukaryotic, preferably mammalian, cells. The invention provides recombinant expression constructs encoding senescence-triggering peptides (STPs) that inhibit cell proliferation and increase heterologous protein production concomitantly. In preferred embodiments expression of STPs by said recombinant expression constructs is inducible in the cell by contacting the cell with an inducer to which an expression control element that controls expression of the STS is responsive.

In one aspect, the present invention provides cell lines comprising senescence-triggering peptides (STPs), most particularly encoded by recombinant recombinant expression constructs introduced into said cells. In certain embodiments, the cell line is a fusion-partner cell line useful for producing monoclonal antibodies. In specifically-provided embodiments, the cell line contains an recombinant expression construct, wherein the recombinant expression construct advantageously encodes STPs. Each recombinant expression construct also preferably contains an inducible transcription regulation element allowing conditional control of the expression of the STPs.

In particular embodiments the cell line is a Sp2/0-Ag14 Mouse B cell myeloma (CRL-1581); a YB2/0 Rat B lymphoblast (Accession no. CRL-1662); a K6H6/B5 Human B lymphoma/Mouse myeloma (CRL-1823); a NS1 Human lymphoblast (CRL-8644); a FO Mouse myeloma (CRL-1646); a Y3-Ag 1.2.3 Rat myeloma (No. CRL-1631); or a P3×63-Ag8-653 myeloma cell line (CRL-1580; all obtained from the American Type Culture Collection, Manassas, Va.) or other suitable fusion-partner cell lines for producing hybridoma.

In certain embodiments, the STPs comprise containing a Cy motif. In certain other embodiments, the STPs comprise an ankyrin-binding motif. In further embodiments, the invention provides a combination of STPs comprising both ankyrin-binding and Cy motifs.

In yet other embodiments, the invention provides recombinant expression constructs containing a selectable marker, and a multiple cloning site flanked by a polyadenylation signal. The inducible transcription regulation element and polyadenylation site are positioned in operable orientation to the STP to be expressed. In certain embodiments, the inducible transcription regulation element contains an SV40 or similar viral promoter and functional operator sequences such as bacterial lac repressor operator.

Another aspect of the present invention provides a method of producing a hydridoma cell comprising fusing a cell of the hydridoma fusion partner cell line with a murine spleen cell.

The invention thus provides methods for increasing heterologous protein production, particularly monoclonal antibody production in a hybridoma, by expressing STPs in said cells.

Specific preferred embodiments of the present invention will become evident from the following more detailed description of certain preferred embodiments and the claims.

### BRIEF DESCRIPTION OF THE DRAWINGS

FIG. 1 is a diagram showing the p53 and Rb pathways that mediate senescence.

FIG. 2 is a diagram of the time course for blocking cell growth following induction of senescence-triggering peptides (STPs), and FIG. 6 is a diagram of a recombinant expression construct (LNtCtx) of the invention, having a multiple cloning site (MCS) and regulatory promoter.

FIG. 3 is a diagram of the enhanced production of secreted monoclonal antibody from hybridoma cells following induction of STPs.

FIG. 4 is a diagram of the enhanced production of secreted alkaline phosphatase from Chinese hamster ovary cells following induction of STPs.

FIG. 5 is a diagram showing the process of producing monoclonal antibodies.

### DETAILED DESCRIPTION OF PREFERED EMBODIMENTS

The present invention relates to cell lines useful for producing proteins and peptides, particularly heterologous proteins and peptides. As used herein, the term “heterologous” is intended to mean a protein not natively made in the cell or encoded in the native chromosomal DNA thereof. Specifically, the term is intended to mean a protein encoded by an exogenous recombinant expression construct introduced into the cell. The term is also intended to encompass production of a monoclonal antibody by a hybridoma cell produced by fusion of an antibody-producing cell with a myeloma cell or other appropriate cell fusion partner known in the art. In certain embodiments, such cell lines can be switched from a replicative to a productive state in which protein biosynthesis is extended. As disclosed herein, said productive state is a premature senescent state. In preferred embodiments the cell line is a hybridoma cell line.

Senescence is defined as the permanent halt in cellular division. Campisi, 2000, In Vivo 14: 183-8. Replicative or cellular senescence was observed and proposed as a model for aging at the cellular level over forty years ago. Hayflick, 1965, Exp. Cell Res. 37: 614-36; Hayflick & Moorhead, 1961, Exp. Cell Res. 25: 585-621. When normal cells are serially cultured, they typically undergo rounds of cell divisions during growth, but as they age in culture the cells lose the capacity to divide. This phenomenon is different from apoptosis, or programmed cell death, and senescent cells are actually resistant to programmed cell death. Indeed, some senescent cells have been maintained in their nondividing state for as long as three years. Smith & Pereira-Smith, 1996, Science 273: 63-7. These cells are very much alive and metabolically active, but they do not divide. This nondividing state has been found to be irreversible by any biological, chemical, or viral agent. At this stage of terminal nondivision, it has been shown by gene expression that the cells have undergone global changes compared to those of their younger counterparts. The relationship between the changes in gene expression and cellular senescence has not been definitively established, and it is not known whether any or all of the changes cause senescence or whether senescence results in the changes in gene expression. Gene expression changes that could potentially induce senescence include a repression of cell-growth-inducing transcription factors. Dimri & Campisi, 1994, Exp. Cell Res. 212: 132-40. However, along with this repression of growth inducers is an activation of the cell cycle inhibitors, p21 and p16, which are more likely the genes that act to induce cell senescence and in fact are the end products of genetic programs that lead cells to senescence. Smith & Pereira-Smith, 1996, Id.

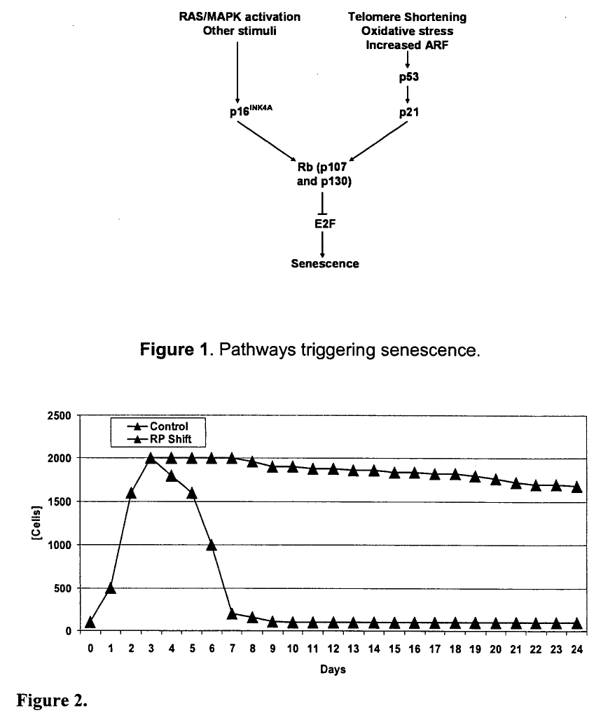

Two separate pathways mediate premature senescence (FIG. 1). Expression of cellular proteins p53 and Rb are activated by various stimuli, including telomere shortening, certain forms of DNA damage, and p14^{ARF} expression (which in turn results from oncogene activation). Increased p53 expression causes a p21-dependent form of growth arrest. Expression of p21 inhibits phosphorylation of Rb family members, resulting in repression of E2F activity that promotes senescence. Expression of p16^{INK4a} is increased by telomere-independent signals, such as MAP kinases. Other stimuli that induce (or inhibit repression of) p16^{INK4a} are not fully characterized. Expression of p16^{INK4a} likewise inhibits Rb phosphorylation with attendant induction of senescence.

Attempts to induce senescence by activating only one of these pathways have yielded results insufficient for long-term cell culture. Expression of p16 alone has been shown to produce a reversible phenotype similar to senescence (termed quiescence in the art for distinction) following cell cycle arrest. Dimri et al., 2000, Molec. Cell Biol. 20: 273-85; Uhrbom et al., 1997, Oncogene 15: 505-14; Vlach et al., 1996, EMBO J. 15: 6595-604. These cells overcome cell cycle arrest and begin proliferating in 7-10 days. There are three potential reasons for this reversal of cell cycle inhibition; 1) the cells have stopped producing p16, 2) p16 is inactivated, 3) p16 is degraded. It is unlikely that the cells halted synthesis of p16 because its expression is driven by a powerful eukaryotic promoter.

Turning to the other possibilities, one of skill in the art will appreciate that the INK4 family of CKIs includes four structural proteins (p15, p16, p18, and p19), each of which contains four ankyrin repeats. Hirai et al., 1995, Molec. Cell Biol. 15: 2672-81. INK4 proteins bind to monomeric CDK4/6 subunits through these ankyrin repeat motifs (Sheaff & Roberts, 1995Curr Biol. 5: 28-31; Serrano et al., 1993, Nature 366: 704-7; Kamb, 1994, Cold Spring Harb. Symp. Quant. Biol. 59: 39-47) as shown in TABLE 1, preventing their association with D-type cyclins, and INK4 proteins also can inhibit the activity of cyclin D-CDK4/6 complexes. Hirai et al., 1995, Molec. Cell Biol. 15: 2672-81; Quelle et al., 1995, Oncogene 11: 635-45. Cells that become resistant to p16 may elevate their levels of CDK4 or CDK 6, thus effectively titrating the p16 produced. Alternatively, INK4 proteins have half-lives of about 20 min in cell culture. Baldin et al., 1993, Genes Develop. 7: 812-21; Quelle et al., 1993, Genes Develop. 7: 1559-71.

TABLE 1
Ankyrin repeat motifs

ANKYRIN III REGION

| | |
|---|---|
| P16 | RPVHDAAREGFLDTLVVLHRA |
| P19 | SPVHDAARTGFLDTLKVLVEH |
| P18 | AVIHDAARAGFLDTLQTLLEF |
| P15 | RPVHDAAREGFLDTLVVLHRA |

(wherein the p16 Ankyrin sequence is identified by SEQ ID NO. 22; p19 Ankyrin sequence is identified by SEQ ID NO. 23; p18 Ankyrin sequence is identified by SEQ ID NO. 24; and p15 Ankyrin sequence is identified by SEQ ID NO. 25). The consensus Ankyrin sequence is: Xaa-Xaa-Xaa-His-Asp-Ala-Ala-Arg-Xaa-Gly-Phe-Leu-Asp-Thr-Leu-Xaa-Xaa-Leu (SEQ ID NO. 5)

Expression of p21 alone produces a more stable senescence-like phenotype than expression of p16 alone (Chang et al., 1999, Oncogene 18: 4808-18; Noda et al., 1994, Exp. Cell Res. 211: 90-8), however, expression of p21 leads to mitotic catastrophe, a slow form of cell death similar to apoptosis. Tsao et al., 1999, J. Virol. 73: 4983-90. p21 has two functional domains, an N-terminal CDK binding region, and a carboxy-terminal region that associates with PCNA, a processing factor for DNA polymerase delta. Flores-Rozas et al., 1994, Proc. Natl. Acad. Sci. USA 91: 8655-9; Harris et al., 1995, J. Clin. Immunol. 15: 232-41; Waga et al., 1994, Nature 369: 574-8. It has been proposed that the N-terminal region including the cyclin binding Cy motif of the CIP/KIP family of CDK inhibitors (Chen et al., 1996, Molec. Cell Biol. 16: 4673-82) can interact with the cyclins independently of CDK2. The cyclin-binding motifs of p21 are required for the optimal inhibition of cyclin-CDK kinases in vitro and for growth suppression in vivo.

Blocking the turnover of these proteins using an inhibitor of the ubiquitin-targteing proteosome, MG132 (Hunt et al., 1999, Biochim. Biophys. Acta 1444: 315-25) sustains the levels of INK4 protein thereby enhancing the cell cycle arrest produced by premature senescence.

Alternatively, it is contested that fragments of Cip/Kip and INK4 proteins deficient in proteasome targeting sequences are stable in cells by being refractory to proteasome degradation.

An alternative approach to expressing full-length cDNA of p 16 and p21 is to express fragments of these genes that may reduce the likelihood of degradation by ubiquitin-targeting or inactivation by binding of cyclin-CDKs. For instance, the cyclin binding motif Cy motif of the CIP/KIP family of CDK inhibitors (Chen et al., 1996, Molec. Cell Biol. 16: 4673-82) can interact with the cyclins independently of CDK2. The cyclin-binding motifs of p21 are required for the optimal inhibition of cyclin-CDK kinases in vitro and for growth suppression in vivo. Peptides containing only the N-terminal or C-terminal motif of p21 partially inhibit cyclin-CDK kinase activity in vitro and DNA replication in Xenopus egg extracts. A Cy motif is found near the N terminus of Cdc25A that is separate from the catalytic domain. Saha et al., 1997, Molec. Cell Biol. 17: 4338-45. Mutations in this motif disrupt the association of Cdc25A with cyclin E- or cyclin A-CDK2 in vitro and in vivo and selectively interfere with the dephosphorylation of cyclin E-CDK2. A peptide based on the Cy motif of sequence of p21 competitively disrupts the association of Cdc25A with cyclin-CDKs and inhibits dephosphorylation of the kinase. p21 inhibits Cdc25A-cyclin-CDK2 association and dephosphorylation of CDK2. Conversely, Cdc25A associates with cyclin-CDK and protects it from inhibition by p21. Cdc25A also protects DNA replication in Xenopus egg extracts from inhibition by p21. Thus, cdc25A and p21 compete for binding with cyclin-CDK complexes. The Cy motif sequence is found in many proteins involved in cell cycle dynamics, and the association of cdc25A, p21, cyclins and CDKs is mediated, in part, by the Cy motif. An alignment of Cy motifs is presented in TABLE 2.

TABLE 2
Cy motifs

Cy motif

| | | | | | | |
|---|---|---|---|---|---|---|
| E2F1 | K | R | R | L | D | L |
| E2F2 | K | R | K | L | D | L |
| E2F3 | K | R | R | L | E | L |
| p107 | K | R | R | L | F | G |
| p130 | K | R | R | L | F | V |
| Cdc6 | G | R | R | L | V | F |
| Myl1 | P | R | N | L | L | S |
| Cdc25a | P | R | R | L | L | F |
| p57 | C | R | S | L | F | G |
| p27 | C | R | N | L | F | G |
| p21(N) | C | R | R | L | F | G |
| p21(C) | K | R | R | L | I | F |
| HPV18E1 | K | R | R | L | F | T |
| SSeCKS(1) | L | K | K | L | F | S |
| SSeCKS(2) | L | K | K | L | S | G |
| b3-endonexin | K | R | S | L | K | L |

(wherein the E2F1 Cy motif is identified by SEQ ID NO. 6; the E2F2 Cy motif is identified by SEQ ID NO. 7; the E2F3 Cy motif is identified by SEQ ID NO. 8; the p107 Cy motif is identified by SEQ ID NO. 9; the p130 Cy motif is identified by SEQ ID NO. 10; the Cdc6 Cy motif is identified by SEQ ID NO. 11; the Myt1 Cy motif is identified by SEQ ID NO. 12; the Cdc25a Cy motif is identified by SEQ ID NO. 13; the p57 Cy motif is identified by SEQ ID NO. 14; the p27 Cy motif is identified by SEQ ID NO. 15; the p21(N) Cy motif is identified by SEQ ID NO. 16; the p21(C) Cy motif is identified by SEQ ID NO. 17; the HPV18 E1 Cy motif is identified by SEQ ID NO. 18; the SSeCKS(1) Cy motif is identified by SEQ ID NO. 19; the SSeCKS (2) Cy motif is identified by SEQ ID NO. 20; and the b3-endonexin Cy motif is identified by SEQ ID NO. 21). The consensus Cy motif is identified as: (Lys/Arg)-Xaa-Leu.

A consensus DNA sequence of senescence-triggering ankyrin repeat similar to the region of p 16 (amino acids 1-60), and similar to the Cy motif of p21 (amino acids 1-81), were separately cloned into nucleic acid encoding an alpha helix produced by amino acids 1-40 of the Escherichia coli L7/L12 ribosomal protein (Accession number P02392). Bocharov et al., 1996, FEBS Lett. 379: 291-4. This peptide-alpha helix sequence was, in turn, cloned into a retroviral recombinant expression construct wherein the STP-encoding nucleic acid was under the transcriptional control of a doxycycline-inducible promoter, which construct was transfected with vesicular stomatitis viral DNA into viral packaging cells using conventional calcium phosphate-utilizing techniques. Baldin et al., 1993, Genes Develop. 7: 812-21. After infection into HT1080 E-14 cells that actively produce the convenient marker protein plasminogen activator inhibitor-1 (PAI-1; Kang et al., 1998, Int. J. Cancer 77: 620-5; Wileman et al., 2000, Br. J. Ophthalmol. 84: 417-22), cells harboring the STPs were selected by including a selective agent into the cell culture media. In the present example, expression of STPs was induced by adding doxycycline (a stable derivative of tetracycline) into the media. Following induction, cell proliferation, as monitored by counting cells, was found to be stopped within 24 hours, and proliferation of the cells remained blocked for as long as 30 days (FIG. 2).

Senescence differs from other forms of growth arrest, such as quiescence, in two important ways. First, senescence in somatic cells is thought to be irreversible, and it therefore represents a specialized form of terminal differentiation. Second, it encompasses certain phenotypic alterations such as characteristic morphological changes and the expression of senescence-associated-β-galactosidase (SA-β-Gal) activity. Recently, senescence has been shown to correlate with the establishment of an unusual form of heterochromatin that is present in discrete nuclear foci (called SA-heterochromatic foci (SAHF)). (Dimri et al., 2000, Molec. Cell Biol. 20: 273-85). In aggregate, these data suggest that senescence results from the durable repression of promoters associated with growth. This repression is enforced by the construction of stable heterochromatin-like complexes, the formation of which is directed in part by hypophosphorylated Rb.

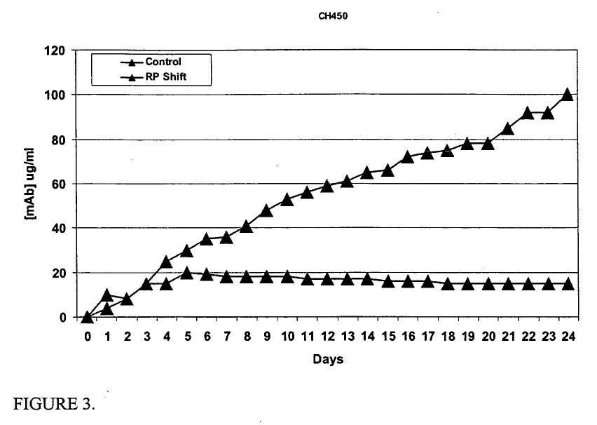

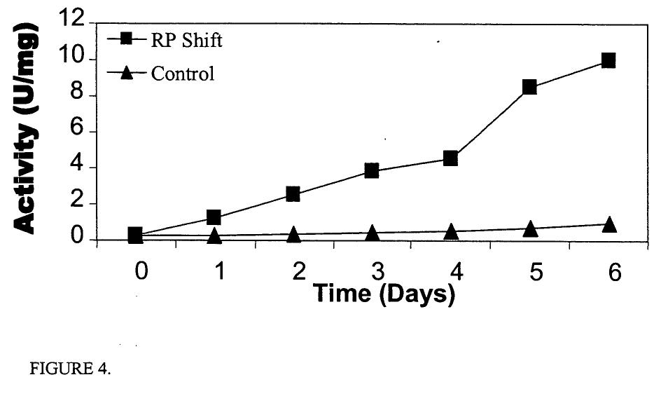

The onset of premature senescence was observed by staining cells for SA-β-gal activity. Dimri et al., 1995, Proc. Natl. Acad. Sci. USA 92: 9363-7. Cells maintained this phenotype for thirty days. Cells to which empty retroviral vector was infected did not express SA-β-gal and displayed a phenotype identical to cells with STPs that were treated with doxycyline. Cells exposed to doxycycline developed a large, flattened appearance, and display SA-β-gal activity; all characteristics associated with the senescence phenotype (Campisi, 2000, In Vivo 14: 183-8) as shown in FIG. 3. The amount of PAI-1 was monitored by ELISA assays in cell culture supernatants from cells with and without inducer, which were frozen at each time-point. PAI concentrations were obtained from a standard curve ranging from 0.01-5 mg/ml. The secreted PAI-protein levels were increased by as much as 30-fold after 8 days of culture with respect to cells not induced with doxycyline as shown in FIG. 4. These results indicate that STPs induce long-term premature senescence in mammalian cells. Similar results have been obtained in other cell lines including CHO and hybridoma cells produced from Sp20 mouse myeloma cells.

In growing cultures, senescence must occur after the cells reach an optimal density for protein production. Proper timing of premature senescence is essential to maximize production after developing the premature senescent state. This proper timing can be achieved by placing the expression of the STPs under the regulation of an inducible promoter. To satisfy this requirement, a retroviral vector with a modified TetR system was developed. Bucciarelli et al., 2003, U.S. Pat. No. 6,635,448; see FIG. 6. This or any repressor system used must prevent expression of the STPs until the culture grows to optimum cell density.

Cell Lines

In certain embodiments, the cell lines of the present invention arrest of cell division by conditionally expressing known blockers of the cell cycle. The stable introduction of the full-length coding regions of cell cycle inhibitor genes or fragments of such genes under control of inducible promoters not only stopped cell division, but induced differentiation to a senescence-like state. As observed for these cell lines, senescence was characterized by an increase in cell volume, a flattened morphology, and increased protein synthesis. Such cells had a longer lifespan and were also substantially more resistant to environmental stresses, such as lowered pH, loss of serum factors, osmotic changes and other impedance that triggers cell death in proliferating population. As a consequence of this phenotypic stability, higher concentrations of secreted products were achieved. As a result, complex media or repetitive exchange of media circumvented. Disclosed herein are mammalian cells, including Chinese hamster ovary (CHO) cells and cell lines commonly used as fusion partners for creating hybridomas, that have been engineered to express cell cycle inhibitor(s) encoded by recombinant expression constructs of the invention, that switch the cells from a replicative to a protein producing premature senescent state (termed “RP Shift” herein).

In certain embodiments, the cells are used at the fusion stage of monoclonal antibody development so that enhanced monoclonal antibody production may be invoked by inducing the RP Shift at the manufacturing stage. The invention is useful in that it allows federal regulatory validation concurrent with the initial investigations of the effectiveness of the mAb.

Fusion partner cell lines, such as SP2/0 and NS1 (TABLE 3), are engineered to contain the STPs under the control of the RP Shift regulatory system. These cells are then fused with mouse spleen cells using standard hybridoma technologies familiar to one skilled in the art. The resulting hybridoma cells are screened for production of monoclonal antibodies that 1) bind to a targeted antigen of interest, and 2) affect the anticipated biological function. After a primary determination of any cohort of hybridoma fusion clones to identify monoclonal antibodies (mAb) having the desired immunological specificity, avidity or other functional property, the RP Shift can be used to produce sufficient quantities of mAb for further investigation and commercialization. Advantageously, STPs encoded by the recombinant expression constructs of the invention are not expressed in said cells until an inducer to which regulatory expression elements controlling expression of the STPs are responsive, such as doxycycline is added to the cell culture medium. Upon induction, cell division is blocked, and they enter a senescence-like state. During this state, the cells produce the expected enhanced quantities of mAbs. The benefit of engineering RP Shift into precursor cell lines is that all cell lines subsequently produced from these precursor cell lines should maintain appreciable similarity in function, growth characteristics, and extent of RP Shift capability.

TABLE 3
Widely used hybridoma fusion partners

| Cell line | Species/cell type | Reference |
|---|---|---|
| Sp2/0-Ag14 | Mouse B cell | Kohler et al., 1978, Eur. |
| A.T.C.C. Accession | myeloma | J. Immunol. 8: 82-8; |
| No. | | Auchincloss et al., 1981, |
| CRL-1581 | | J. Immunol. 127: 1839-43 |
| YB2/0 | Rat B lymphoblast | Kilmartin et al., 1982, J. |
| A.T.C.C. Accession | | Cell Biol. 93: 576-82 |
| No. | | |
| CRL-1662 | | |
| K6H6/B5 | Human B | Carroll et al., 1986, J. |
| A.T.C.C. Accession | lymphoma/Mouse | Immunol. Methods 89: 61-72 |
| No. | myeloma | |
| CRL-1823 | | |
| NS1 | Human lymphoblast | U.S. Pat. No. 4,720,459 |
| A.T.C.C. Accession | | |
| No. | | |
| CRL-8644 | | |
| FO | Mouse myeloma | de StGroth & |
| A.T.C.C. Accession | | Scheidegger, 1980, J. |
| No. | | Immunol. Methods 35: |
| CRL-1646 | | 1-21 |
| Y3-Ag 1.2.3 | Rat myeloma | Galfre et al., 1979, |
| A.T.C.C. Accession | | Nature 277: 131-3 |
| No. | | |
| CRL-1631 | | |
| P3X63-Ag8-653 | Human myeloma | Kohler & Shulman, |
| A.T.C.C. Accession | | 1978, Curr. Top. |
| No. | | Microbiol. Immunol 81: |
| CRL-1580 | | p. 143-8 |

Production of Monoclonal Antibodies

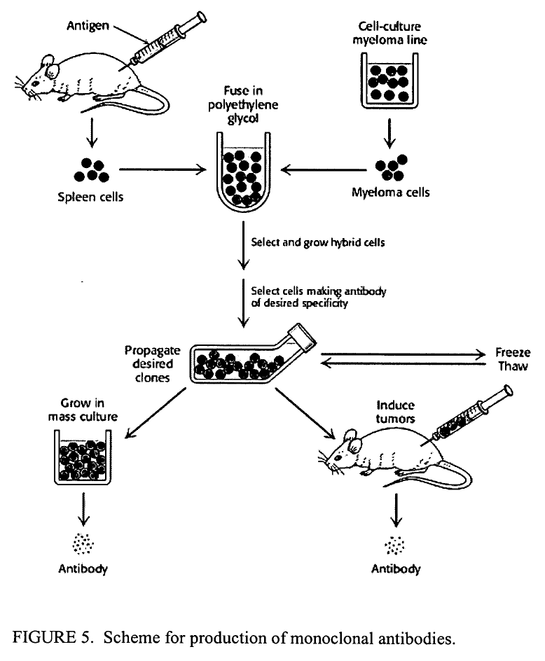

In certain embodiments of the present invention, a fusion partner cell line is used in the production of monoclonal antibodies. The production of monoclonal antibodies requires the steps of: immunizing an animal; obtaining immune cells from the animal's spleen; and fusing the immune cells with a cancer cell (fusion partner), such as cells from a myeloma, to make them immortal. The animal used in normally a mouse, however, other mammals may also be used. The fused cell, termed a hybridoma, secretes monoclonal antibodies. A typical procedure used for producing monoclonal antibodies is illustrated in FIG. 5.

An investigator who wishes to target a particular protein or other molecule associated with a particular disease state generates a cell line that secretes monoclonal antibodies that react strongly with that protein or molecule. One method of manufacturing a cell line that produces monoclonal antibodies is to fuse mouse lymphocytes with an immortalized myeloma cell line (See TABLE 3) known as a fusion partner, to produce monoclonal antibody bearing hybridomas. In order to achieve large-scale production of monoclonal antibodies, however, hybridoma cells must grow and multiply to form a clone that will produce the desired monoclonal antibodies. On an industrial scale, the method of choice for growing these cells is large bioreactors containing cell-culture medium. This technique requires some expertise, requires special media, and can be expensive and time-consuming. There has been considerable research on in vitro methods for growing hybridomas and these newer methods are less expensive, are faster, and produce antibodies in higher concentration than has been the case in the past.

Use of Transgenic Animals in the Production of Monoclonal Antibodies

In other embodiments of the present invention, transgenic animals used for producing monoclonal antibodies. Laboratory and domestic animal transgenesis has enormous potential, inter alia, to create transgenic animals as bioreactors for the production of useful proteins such as therapeutic proteins and antibodies. Transgenic animals (mice, for example) were first generated by in vitro microinjection of a fertilized egg with the foreign DNA gene, implantation into pseudopregnant foster mothers and identification of transgenic progeny (for example, by PCR). In mice, the development of embryonic stem cell technology has simplified the initial step because these cultured cells can be easily transfected, manipulated in vitro and then implanted into a multicellular blastocyst, leading to transgenic animals. Homozygous progeny can be generated from the crosses of the chimeric individuals produced from these transgenic animals.

There are many examples of the application of transgenic mouse technology in the field of recombinant immunoglobulins, to create animals that constitutively produce recombinant antibodies or antibody fragments capable of neutralizing common pathogens. Animal transgenesis can also be used to create mice that carry human variable and constant gene segments in germline configuration. These animals produce rearranged human antibodies in their B cells and produce human antibodies after conventional immunization procedures. Glockshuber et al., 1990, Biochemistry 29: 1362-7; Mendez et al., 1997, Nature Genet. 15: 146-56; Neuberger & Bruggemann, 1997, Nature 38: 25-6; Yang et al., 1999, Cancer Res. 59: 1236-43. Transgenic mouse strains that produce high-affinity human monoclonal antibodies after immunization with antigens, including human antigens, using conventional hybridoma techniques are obtained when large size (mega-base) human genomic DNA fragments containing heavy or light chain genes were introduced into murine embryonic stem cells. These cells are then used for blastocyte injections. The resulting transgenic mice are crossed with mice having disrupted endogenous heavy and light (k) chain loci to produce mice having human antibody chain-encoding genes.

These transgenic mice produce human IgM and IgG antibodies at relatively high serum concentrations. The antibodies are composed of a high proportion of human k (not mouse k) and the usage/patterns of heavy and light chain V, D, and J germinal genes are similar to what is found in human peripheral blood lymphocytes. Both the length of CDR3 and N-addition are characteristically human. Abgenix (Freemont, Calif.) (Xenomouse) and Medarex (Princeton, N.J.) (HuMAb-Mouse) have developed transgenic mouse strains that produce antigen-specific human monoclonal antibodies using conventional hybridoma technology. Hybrids developed in this way are stable and secrete large amounts of high-affinity (dissociation constant in the order of 10⁻⁹ M) antibodies.

Antibodies produced by these mice have the limitation, however, that they express a mouse-specific glycosylation pattern (Borrebaeck, 1999, Nature Biotechnol. 17: 621) that poses a potential risk to using these “human” antibodies therapeutically. Thus, CHO cells, which have a pattern of glycosylation more similar to that of human cells, may be preferable for recombinant “humanized” antibody production.

Types of Therapeutic Antibodies

Various embodiments of the present invention provide recombinant cells that permit chimeric hybrid immunoglobulins, humanized hybrid immunoglobulins, and recombinant antibody immunoglobulin fragments to be produced at useful yields. Chimeric hybrid immunoglobulins retain the original murine variable regions and the constant regions are switched for those of a human antibody to try to reduce HAMA and gain human effector functions. These antibodies are used in therapy when effector and other Fc-associated functions and properties are needed. Humanized hybrid immunoglobulins possess murine residues that conform to specific complementarity antibodies determining regions and others of possible structural relevance are “transplanted” to a human antibody framework. From here, the corresponding regions and residues have been eliminated to try to abrogate HAMA and gain human effector functions. These antibodies are used in therapy when effector and other Fc-associated functions and properties are needed.

Recombinant immunoglobulin fragments also may be produced in bacteria or yeast. These fragments include Fabs, fragments Fv and engineered Fv (scFv, dsFv), their variants minibodies, CRAbs, multifunctional and multi-specific diabodies, triabodies, tetrabodies) and fusion constructions (immunodrugs, immunotoxins, BRM). Recombinant antibody fragments have been used for in vivo radioimmunodetection and in situ radiotherapy, drug, toxin and BRM-targeted delivery, detoxification of drugs and toxins, direct or indirect neutralization of viruses and microorganisms, homogeneous diagnostic assays and catalysis. These antibody fragments are also advantageously produced using the cells provided herein.

Cloning of Antibody V-Region Genes for Recombinant Antibodies

In other embodiments, cell lines of the present invention are used for producing recombinant antibodies. Important immunoglobulin DNA sequences to be cloned and manipulated for antibody engineering to proceed are the variable (V) domain genes of both heavy (H) and light (L) chains that determine the complete functional structure of the antigen-binding site. The initial cloning of immunoglobulin genes involved the construction of libraries of genomic DNA (Morrison, 1985, Science 229: 1202-7), from donor cells (hybridomas, B-lymphocytes or B-tumor cell lines). This procedure is slow and tedious because it is necessary to identify clones containing rearranged, complete antibody genes. Introns, existing between the signal peptide, V and constant (C) domain-encoding sequences, imposed additional technical difficulties.

The development of the polymerase chain reaction (PCR; Mullis et al., 1986, Cold Spring Harb. Symp. Quant. Biol. 51: 263-73), and its application to the field (Larrick et al., 1988, Prog. Clin. Biol. Res. 272: 383-93; Larrick et al., 1989, Biochem. Biophys. Res. Commun. 160: 1250-6; Orlandi et al., 1989, Proc. Natl. Acad. Sci. USA 86: 3833-7), greatly simplified the cloning of V region genes and set the stage for extraordinary advances in antibody engineering. To amplify the VL and VH immunoglobulin regions by PCR, cDNA is fabricated by reverse transcription from RNA extracted from hybridomas or B-lymphocytes. With the amplification of the desired mature V-region genes, this step avoids completely the interference by rearranged genes and introns. The PCR primer sets are designed on the basis of conserved flanking base sequences that exist at the beginning of VH and VL genes (corresponding to the FR1 in the V region), at the end of the JH or JL segments (FR4 in the rearranged V region) and at the proximal CL or CH1 constant regions. Larrick et al., 1989, Id.

Because different V and J germinal genes probably evolved from a single precursor and have strong sequence similarities-combined with the possibility of introducing degenerate positions in the primer—the number of synthetic oligonucleotide pairs required to prime at the 5′- and 3′-ends of any given V region can be reduced to a minimum. Many example primer sets for the PCR cloning of V regions can be found by searching the literature. The development of antibody combinatorial library technology boosted these efforts and has produced several reliable sets of PCR primers for both mouse and human V regions. de Haard et al., 1999, J. Biol. Chem. 274: 18218-30; Kettleborough et al., 1993, Eur. J. Immunol. 23: 206-11. When designing antibody PCR primers, other characteristics to bear in mind include particular restriction enzyme sites and tags; expression strategies and specific vectors; and detection and purification strategies. In the case of mouse hybridomas, special care must be taken when designing the synthetic oligonucleotides to avoid the amplification of an aberrant kappa RNA transcript that is present in many of these cells, inherited from the original MOPC-21 tumor from which most myeloma fusion partners are derived. Carroll et al., 1988, J. Exp. Med. 168: 1607-20.

Conditions for successful antibody PCR have improved tremendously since the first reports. Now available are RNA extraction and cDNA kits, error-free thermostable DNA polymerases, designer reaction-buffer compositions and other PCR paraphernalia, from sophisticated thermocyclers to custom reaction tubes. Also, the large amount of accumulated information on new antibody sequences has decisively contributed to the explosive use of this simple, powerful technique.

Chimeric and Humanized Recombinant Antibodies

In yet further embodiments, cell lines of the present invention are used to produce chimeric and humanized recombinant antibodies. In the early 1980s, several groups developed the basis of what is now known as antibody genetic engineering. Gillies et al., 1983, Cell 33: 717-28; Neuberger, 1983, EMBO J. 2: 1373-8; Oi et al., 1983, Proc. Natl. Acad. Sci. USA 80: 825-9; Sharon et al., 1984, Nature 309: 364-7; Wood et al., 1985, Nature 314: 446-9. With the pressing problem of an HAMA response after murine MAb treatment and the lack of desired effector functions, it is not surprising that the first recombinant therapeutic antibodies were prepared with a simple substitution of the murine constant domains by similar ones of human origin (Boulianne et al., 1984, Nature 312: 643-6; Morrison et al., 1984, Proc. Natl. Acad. Sci. USA 81: 6851-5; Neuberger et al., 1984, Nature 312: 604-8), the contents of which are incorporated by reference. To fabricate these chimeric antibodies, the V regions of the therapeutic murine antibody can be cloned by PCR and inserted into vectors carrying human gamma and kappa (or lambda) constant domains. These vectors can be prepared to include both chains for tandem expression, or they can be produced as separate plasmids for the heavy and light chains. Different host cell lines have been transfected with these chimeric genetic constructions using electroporation, liposome fusion or the calcium chloride method. Recipient cells include myelomas, Chinese hamster ovary (CHO) cells and insect cells. After seeding in a selective medium where only transfectomas will grow, the cultures are screened and cloned in a manner similar to conventional hybridomas. Among host cells, CHO cells are preferred because of their more human-like glycosylation pattern and adaptability for high-density, large-scale cultivation in serum and protein-free media, an important aspect for production (Reff, 1993, Curr. Opin. Biotechnol. 4: 573-6), the contents of which are incorporated by reference. Compared to hybridomas, transfectomas secrete much lower amounts of antibodies and frequently lose antibody secretion. One approach to solving this problem has been to use the dihydrofolate reductase (DHFR; Hendricks et al., 1988, Gene 64: 43-51), the contents of which are incorporated by reference, selection and amplification system as part of the vectors to ensure multicopy integration and much higher antibody expression.

While this selection is effective in CHO cells, its use becomes more complex in myeloma cells because of endogenous DHFR activity. An alternative vector system for high expression of recombinant antibodies in cells of myeloid origin has been developed based on glutamine synthetase as a selectable marker and the almost absolute dependence of myelomas and hybridomas on exogenous glutamine for in vitro growth (Bebbington et al., 1992, Biotechnology (NY) 10: 169-75), the contents of which are incorporated by reference. Chimeric antibodies show the same specificity and affinity of parental murine antibodies and are capable of efficiently mediating ADCC, antibody dependent macrophage cytotoxicity (ADMC) and complement fixation in the human context. In general, substitution of the murine constant regions by the human ones increases the biological half-life in vivo.

Many chimeric versions of potential therapeutic murine antibodies have been evaluated in clinical trials (Khazaeli et al., 1994, J. Immunother. 15: 42-52), the contents of which are incorporated by reference. From these studies, it has become clear that, while in some cases chimerization caused a total disappearance of the HAMA response, many chimeric antibodies remained immunogenic as a result of the presence of the murine V regions. Another way of reducing the immunogenicity of murine V regions was reported by Mateo et al. (1997, Immunotechnology 3: 71-81), the contents of which are incorporated by reference. These authors mutated mouse V region T-cell epitopes to the homologous human sequences, leaving the complementarity determining regions (CDRs) and the Vernier zone untouched. The antimouse response in primates was nullified by the chimeric antibodies produced by linking the remodeled mouse V regions to human constant domains.

The first fully humanized (reshaped) antibody (CAMPATH-1H) was produced by Riechmann et al. (1988, Nature 332: 323-7, the contents of which are incorporated by reference) using the anti-CD52 CAMPATH-1 rat antibody as target and the CDR-grafting technique originally proposed by Winter and colleagues (Verhoeyen et al., 1988, Science 239: 1534-6). CDR-grafting involves the synthesis of a completely artificial V region using sequence information from the CDRs from the murine therapeutic antibody, combined with a compatible human framework (FR) sequence. The new, humanized V regions are linked to suitable human constant regions for expression of the complete immunoglobulin. In their approach, Winter and coworkers proposed using the same human V region for the needed framework. If grafting the murine CDRs reduced or abolished the binding affinity, new constructs are prepared to incorporate additional mouse residues near the CDRs until original binding characteristics are restored. Graham et al., 1995, J. Chem. Technol. Biotechnol. 63: 279-89. This work was soon followed by that of the Protein Design Labs group (Fremont, Calif., USA), which was also based on CDR-grafting. Co et al., 1991, Proc. Natl. Acad. Sci. USA 88: 2869-73. However, the parental murine V region sequences are first matched to the most similar human ones using available databases. Computer modeling is then employed to identify those few murine residues that make key contacts with the CDRs. Rodent amino acids at these positions are then introduced into the human framework, together with the CDRs themselves. While these engineered antibody molecules should not be considered fully, humanized immunoglobulins undisputedly have shown far less HAMA response in clinical trials compared to their chimeric siblings. Rebello et al., 1999, Transplantation 68: 1417-20; Woodle et al., 1999, Transplantation 68: 608-16. They now account for a substantial percentage of immunoglobulin products in ongoing clinical trials.

Immunogenic epitopes can still be present in the reshaped V regions as a result of somatic mutation. Biovation offers the so-called “Delmmunisation” technology to remove immunogenic epitopes, even from CDRs, where appropriate. Forster et al., 1994, Molec. Biotechnol. 1: 251-263.

Other Antibody Constructions

In another embodiment, antibodies are produced as genetic fusion proteins with toxins, drugs, enzymes and other functional groups and modified in their constant domains to alter the original effector mechanisms and properties of the antibody molecule. Such modified antibodies have been produced using conventional hydridoma technology. Harvill et al., 1996, J. Immunol. 157: 3165-70; Penichet et al., 1999, J. Immunol. 163: 4421-6; Sensel et al., 1997, Chem. Immunol. 65: 129-58 Immunoadhesins (Chamow & Ashkenazi, 1996, Trends Biotechnol. 14: 52-60) are fusion proteins that combine the hinge and Fc regions of an antibody with domains of a ligand-specific cell surface receptor. These molecules have advantages as laboratory experimental tools and as promising applications in medicine. Antigenized antibodies (Xiong et al., 1997, Nature Biotechnol. 15: 882-6) are made by grafting peptide epitopes derived from antigens that are different from immunoglobulins in the place of the CDR loops of an immunoglobulin. The conformationally restricted exposure of short foreign peptides using the V region framework and the characteristics of the constant antibody domains create promising combinations for immunoprophylaxis or immunotherapy. They can also be extended to the peptide hormone field and the rational design of new drugs. Finally, Lunde et al. (1999, Nature Biotechnol. 17: 670-5) have recently engineered anti-IgD antibodies with T-cell epitopes inserted into a constant region. This improves the efficiency of B cells to present antigens and stimulates specific T cells, an approach that may be useful in generating new vaccines.

Antibody Fragments

In another embodiment, antibody fragments are produced. Antibody fragments have identical specificity and affinity to the parent antibody molecule. They can be produced with similar or increased avidity (multivalent fragments) or with half the avidity of the whole antibody molecule (monovalent fragments). Antibody fragments (Fab, F(ab)₂ and Fv) have potential advantages over whole antibodies for many therapeutic uses because of their smaller size and potentially better tissue penetration and clearance. Dall'Acqua & Carter, 1998, Curr. Opin. Struct. Biol. 8: 443-50. Fab (about 50 kDa) and Fv (about 25 kDa) antibody fragments are rarely glycosylated, a fact that favors their expression in yeast. Research on production of recombinant antibody fragments in yeast was stimulated by their properties and potential applications and also because of the difficulties in controlled large-scale production of fragments by enzymatic means. The first reports of antibody fragments produced in yeast appeared more than 10 years ago. Horwitz et al., 1988, Proc. Natl. Acad. Sci. USA 85: 8678-82. This work included the so-called single-chain Fv fragments (scFv), artificial constructions where the individual heavy and light chain V regions are linked into a single protein using a short hydrophilic and flexible polypeptide linker (10-20 residues; Horwitz et al., 1988, Proc. Natl. Acad. Sci. USA 85: 8678-82) made of amino acids that will not interfere with the packing of the hydrophobic interfaces of the VH-VL heterodimer. ScFvs are stable at 37° C., retain the specificity and affinity of the original Fv (about 27 kDa) and are easier to express when compared with recombinant Fab, where two separate proteins must be expressed and then fold together. Also, scFv genes can be easily generated by PCR. Davis et al., 1991, Biotechnology (N Y) 9: 165-9.

Antibody fragments have also been expressed in fungi (Davis et al., 1991, Biotechnology (N Y) 9: 165-9), mammalian cells (Riechmann et al., 1988, J. Molec. Biol. 203: 825-8) and insect cells (Kretzschmar et al., 1996, J. Immunol. Methods 195: 93-101). Production of antibody fragments from yeast cells (Romanos et al., 1992, Yeast 8: 423-88) merits special mention. Antibody fragments have been expressed in Saccharomyces cerevisiae (Kotula & Curtis, 1991, Biotechnology (NY) 9: 1386-9) Scizosaccharomyces pombe (Kotula & Curtis, 1991, Biotechnology (NY) 9: 1386-9) and most recently in Pichia pastoris. Eldin et al., 1997, J. Immunol. Methods 201: 67-75; Ridder, 1995, Biotechnology (NY) 13: 255-60. The last appears to be a system with excellent production characteristics and combines the general features of high capacity of secreted antibody fragment expression with fast growth and low levels of contaminant proteins in the medium. Ridder, 1995, Biotechnology (NY) 13: 255-60. The field of recombinant antibody fragments is probably where imagination has most expanded the range of potential products. Mono- and multivalent fragments of many different types, single or bispecific binding activity, genetic coupling to other proteins and functional groups and, more recently, single domain antibodies of different species origin are among the constructions tested to date. Hudson & Kortt, 1999, J. Immunol. Methods 231: 177-89; Hudson, 1999, Curr. Opin. Immunol. 11: 548-57; Riechmann & Muyldermans, 1999, J. Immunol. Methods 231: 25-38. Antibody fragments are being developed for in vivo radioimmunodetection and in situ radiotherapy (Kettleborough et al., 1993, Eur. J. Immunol. 23: 206-11; Adams & Schier, 1999, J. Immunol. Methods 231: 249-60; Mayer et al., 1999, J. Immunol. Methods 231: 261-73; drug (Huennekens, 1994, Trends Biotechnol. 12: 234-9), toxin (Kreitman et al., 1999, Blood 94: 3340-8; Pastan & Kreitman, 1998, Adv. Drug Deliv. Rev. 31: 53-88) and biological response modifier (BRM)-targeted delivery. Melani et al., 1998, Cancer Res. 58: 4146-54.

Preparation of RP Shift Cell Lines Competent for Premature Senescence

Cells useful in the production of recombinant proteins, particularly antibodies, more particularly monoclonal antibodies and therapeutic antibodies especially, or antibody-like proteins or polypeptides, such as those listed as fusion partners in Table 1, CHO, NS0, or other eukaryotic cell lines can be prepared by transfection or infection of a vector harboring STPs into actively growing cells.

Recombinant Expression Constructs for Expressing STPS

The present invention provides recombinant expression constructs allowing tight transcriptional regulation of an encoded polypeptide or STP. Such constructs are useful in methods of causing a premature senescence in a cell transfected, transformed, or infected with the construct.

In certain embodiments, cells are produced or maintained that contain one or more recombinant expression constructs encoding one or more proteins that contribute to the induction of a premature senescent state in the cells.

In some embodiments, the cell line contains an recombinant expression construct encoding one or a plurality of Cy protein motifs, or one or a plurality of ankyrin-binding protein motif, or a plurality of both Cy protein motifs and ankyrin-binding motifs.

The recombinant expression construct may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs conventional ligation techniques that are known to the skilled artisan.

Recombinant expression constructs may contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2 micron plasmid origin is suitable for yeast, and various viral origins (SV40, polyoma, adenovirus, VSV or BPV) are useful for cloning vectors in mammalian cells. However, in mammalian systems, it is preferred that the construct integrate into the genome thereby becoming dependent on the host for replication. Thus, in certain embodiments, the construct comprises a retrovirus-based vector.

Recombinant expression constructs will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

Recombinant expression constructs usually contain a promoter operably linked to the polypeptide-encoding nucleic acid sequence to direct mRNA synthesis. Promoters recognized by a variety of potential host cells are well known. Promoters suitable for use with prokaryotic hosts include the β-lactamase and lactose promoter systems, alkaline phosphatase, a tryptophan (trp) promoter system, and hybrid promoters such as the tac promoter. Promoters for use in bacterial systems also will contain a Shine-Dalgamo (S.D.) sequence operably linked to the polypeptide encoding region.

Examples of suitable promoting sequences for use with yeast hosts include the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Transcription from recombinant expression constructs in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription in higher eukaryotes may be increased by inserting an enhancer or repressor sequence into the construct. Enhancers and repressors are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers.

In certain embodiments, a recombinant expression construct of the present invention contains a senescence-responsive element to increase the production of a recombinant protein. Additional amplification of the desired recombinant protein is achieved by engineering a senescence-responsive element into the vector upstream of a viral promoter, such as the CMV promoter. A senescence-responsive element has been defined at the −89 to −66 sequence (5′-AGGATGTTATAAAGCATGAGTCA-3′ (SEQ ID NO:2)) of the human collagenase gene. Development of the senescence phenotype by expression of the STPs activates senescence-specific transcription factors, thereby further accelerating transcription of the recombinant protein of interest. In certain embodiments, the senescence-responsive element may be operably connected to a bicistronic construct comprising a combination of desired recombinant product and the IRES-driven cell cycle inhibitors separately or both transcribed from the regulated promoter. Such a bicistronic design provides simultaneous regulated expression of the target protein and the cell cycle regulator.

In certain embodiments, an recombinant expression construct of the present invention contains elements that allow tight regulation of gene expression. For example, the recombinant expression construct may contain one or more tetracycline repressor binding sites (tetracycline operators) in the promoter region of the vector. In certain embodiments, the construct comprises multiple tetracycline operators and a minimal promoter comprising a TATA sequence. Preferably, the tetracycline operators are arranged to provide tight regulation of the promoter. One such arrangement includes two phased tetracycline operators 21 basepairs downstream from the TATA sequence and two phased tetracycline operators 11 basepairs upstream from the TATA sequence.

When constructs comprising tetracycline repressor binding regions are used, it is necessary to deliver the tetracycline repressor into the cells chosen for biopharmaceutical production. The tetracycline repressor may be introduced into these producer cells via a retroviral transduction using IRES-containing single-transcript vector. After these producer cells are modified to express tetracycline repressor, the tetracycline-regulated construct containing the CKI is integrated into the genome of the producer cells by retroviral infection. Cells harboring the RP Shift vector as stable transductants may be selected by resistance to the antibiotic G418. The expression of the delivered CKI or other cell-cycle inhibitor may then be induced by adding doxycycline (a stable derivative of tetracycline) into the media.

Recombinant expression constructs used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the polypeptide.

The RP Shift construct useful for expressing STPs is described in U.S. Pat. No. 6,635,448, the contents of which are fully incorporated for all purposes by this reference. The recombinant expression construct, must be (1) tightly regulated, to allow robust cell growth when in the OFF position; (2) highly inducible by inexpensive and FDA-approved ligand; and (3) very promiscuous to allow efficient incorporation and subsequent expression in a wide variety of cells. For these reasons, the tetracycline-regulated retroviral vector is the system of choice. This vector was built from T-REx plasmid vector (Invitrogen) with several modifications. Its final form contains expressed from viral long terminal repeat (LTR) (1) a selection marker (2) and a multiple cloning site (3) flanked by a polyadenylation signal (4) and a regulated promoter (5).

cDNAs for the CKIs, in sense orientation, or other inhibitors of the cell cycle (see following sections), are cloned into the multiple cloning site (MCS) to be expressed from the regulated promoter (5). The regulated promoter and polyadenylation site are positioned in reverse orientation to the LTR to prevent read-through from the LTR, thereby eliminating LTR-initiated expression leakage. At the same time reverse orientation of polyadenylation signal does not interfere with genomic RNA transcription in packaging cells.

Advantageous modifications of the T-REx vector include insertion of two phased tetracycline operators 21 bp downstream from the TATA site and two phased tetracycline operators 11 bp upstream of the TATA sequence within the CMV promoter. This configuration positions a tight protein clamp of two dimerized tet-repressors both in front of the TF-IID contact site and also exactly at the site of initiation of transcription. Moreover, binding of dimerized tetracycline repressors induces a significant kink in the double helix, further reducing the probability of fortuitous transcription.

The second component of the system is the tetracycline repressor, modified to incorporate a nuclear localization signal. Triggering the senescence phenotype by expression of the STPs should activate as yet undetermined senescence-specific transcription factors, thereby accelerating transcription of the recombinant protein of interest. Experiments testing several modifications of the regulated cassette, including a combination of desired recombinant product and the IRES-driven cell cycle inhibitors separately or both transcribed from the regulated promoter. Such a dicistronic design provides simultaneous regulated expression of the target protein and the cell cycle regulator.

Preparation of Hybridoma Cells Containing Inducible STPS

Production of mAb is carried out by immunization of BALB/c mice with three intraperitoneal injections, at 2-week intervals, of purified CAP. Purified CAP is emulsified in the same amount of Freund's complete adjuvant for the first injection and in Freund's incomplete adjuvant for the following two booster injections. Finally, 3 days before the fusion experiment, the antigen is injected intravenously without adjuvant. The fusion of murine spleen cells and myeloma cells (e.g., P3×63-Ag8-653; ATCC CRL 1580, see Table 1) is carried out as described (Kohler, et al, 1978, Eur J Immunol, 8: 82-8).

In brief, the immunized mouse is terminated and the spleen is removed aseptically. The spleen cells were then mixed at a ratio of 5:1 with myeloma cells growing at the logarithmic phase. The cells are fused in the presence of 0.5% polyethylene glycol (PEG 1500; Boehringer Mannheim GmbH, Mannheim, Germany) while being maintained in a 37° C. water bath. The fusion products are diluted in 40 ml of complete Dulbecco's Modified Eagle medium containing 10% fetal bovine serum and are plated out at 100 μl per well in four 96-well plates. After 24 h of incubation, 100 μl of selective medium containing hypoxanthine, aminopterin, and thymidine (HAT) is added to each well. Two more HAT changes are made at 3-day intervals. After this the cells are grown in hypoxanthine and thymidine medium for the next 2 weeks with frequent changes of the same medium. Aliquots of medium from wells with growing hybridomas are screened for the production of antibodies against CAP by enzyme-linked immunosorbent assay (ELISA). Positive hybrids are subcloned by limiting dilution in 96-well plates, and hybridomas are selected for further study and production.

Samples of the chosen hybridoma cell lines are tested by triggering senescence-associated beta galactosidase activity by adding 2 μM of the inducer doxycyline (Dimri, G. P., et al., 1995, Proc Natl Acad Sci US A,. 92: p. 9363-7). Cell lines are examined for mAb production using standard sandwich ELISA techniques.

Although the present invention has been described with reference to particular embodiments, it is to be appreciated that various adaptions and modifications may be made without departing from the spirit and scope of the invention. The invention is only to be limited by the appended claims.

### EXAMPLE 1

### Premature Senescence Enhances Protein Secretion in Hybridoma Cell Lines

Experiments were designed to determine the robustness of premature senescence methods in low, medium and high mAb producing cell lines. An important concern for mAb development is to produce enough mAb to complete preclinical and early clinical studies. Often, hybridomas secreting high affinity mAbs produce low titers of mAbs. These hybridoma cell lines are typically excluded from further product development because of a lack of mAb necessary to complete the studies. Premature senescence can enhance the production capacity of these low mAb producers, so that these more effective antibodies may continue in therapeutic development. Therefore, the hybridoma target cells used in these feasibility investigations were L5G3 producing IgG toward L1CAM, MH70 producing IgG toward rhodopsin, and CH450 producing IgG toward CD24. These hybridoma cell lines were chosen for their different levels of mAb production, LG53 (20 μg/ml), MH70, (100 μg/ml), and CH450 (500 μg/ml). All hybridoma cell lines were grown in Iscove's modified Eagle's medium. To design a senescence-competent cell line, it is necessary to deliver tetracycline repressor (TetR) or other suitable regulatory system into the cells chosen for biopharmaceutical production. As disclosed in more detail herein, the TetR was introduced into these producer cells via a retroviral transduction using IRES-containing single-transcript vector (Levenson, V. V., et al., 1998, Hum Gene Ther,. 9: p. 1233-6). The full-length tetracycline repressor was cloned into a retroviral vector expressing puromycin N-acetyl transferase, LXIP, at EcoRI/XbaI sites. An IRES-containing retroviral construct was used for delivery of the native TetR, engineered to include a nuclear localization signal. This TetR was required to prevent the expression of the senescence-triggering factors in the absence of inducer during growth and preparation of the cell lines.

The Pantropic system was used to deliver the retroviral DNA into the cells. The Pantropic system uses VSV-G, an envelope glycoprotein, to mediate viral entry into cells through lipid binding and plasma membrane fusion (Burns, J. C., et al.,. 1993, Proc Natl Acad Sci US A,. 90: 8033-7 Emi, et al., 1991 J Virol, 65: p. 1202-7 Yee, et al., 1994, Proc Natl Acad Sci USA, 91: p. 9564-8). Because this system does not depend on specific cell surface receptors, the Pantropic System allows transduction of any actively dividing cells. To produce infectious pantropic retroviral particles the vector carrying the gene of interest was transfected into GP2-293 cells using standard Ca-phosphate techniques (Pear, W. S., et al., 1993, Proc Natl Acad Sci USA, 90: 8392-6). Twenty-four hours after transfection, culture medium with infectious virions was collected, filtered through 0.45 μm filter to remove stray packaging cells, supplemented with Polybrene™ (4 μg/ml) and added to 250,000 target cells. Twenty-four hours later cells were trypsinized and re-plated: two 60-mm plates were seeded with five hundred cells each, while the rest of the cells was plated into 150-mm plates at a density of 10⁶ cells per plate. Populations of cells containing TetR were selected with puromycin at an infection rate of 55%. Puromycin selected cells were confirmed to express TetR by reverse-transcription PCR using the Enhanced avian RT-PCR kit (Sigma, St Louis, Mo.) and primers that flank the region 2360-2575 of the TetR (TR1:5′-GGAGGGCAT-GGATGCTAAGTCAC-3′ (SEQ ID NO. 3); TR2:5′-TCTCCCTTCTCCAACCGG AGGATCAC-3′ (SEQ ID NO. 4)).

After hybridoma cells were modified to express TetR, retroviral vectors with inducible tetracycline-regulated promoters containing senescence-triggering factors and the selectable marker for G418 were prepared as follows. Senescence-triggering factors derived from the Ankyrin III region of p16 (Table 1) and the N-terminal Cy region of p21 (Table 2) were synthesized as complimentary oligonucelotides and blunt end cloned into the DNA encoding the N-terminal twenty amino acids of the ribosomal protein L7 from Escherichia coli (SEQ. ID. No. 1) and were cloned into the EcoRI/XbaI site of the retroviral vector described above. Populations of target cells (250,000 cells in 60 mm dishes) were repeatedly (4 times) infected with 4 ml of viral supernatants from GP-293 cells as described above. Cells specifically containing the RP Shift vector harboring inducible STPs were selected with 0.7 mg/ml G418.

The ability of cells to undergo premature-senescence was examined in populations of target cells by addition of the inducer doxycycline to the culture. The relative growth of cells was measured directly by counting cells under the microscope in a hemacytometer (20× phase contrast) at time intervals following addition of media (control) or media containing inducer (treated). Cells were seeded into 12 well plates at 25,000 cells per well. After 48 hrs, doxycycline is added, and cells were trypsinized and removed from triplicate wells every day for three week. Media was changed on day 8 to both control and treated cultures to maintain viability of doxycycline-treated cells. The growth of hybridoma cells treated with inducer was blocked and cell viability remained intact over three weeks, in contract to control cells which died during the first week of culture FIG. 2.

Senescence-competent hybridoma cell lines were isolated by limiting dilution and tested individually for enhanced production of immunoglobulin. Limiting dilution is performed by diluting the population of hybridoma cell cultures so that an average of 1.6 cells is seeded into each well of a 96-well plate. Single colonies were expected to grow in most wells of the plate. Each single colony was recovered from the plate, and grown separately as a cell line. Each cell line was seeded into a single well in each of two identical 96-well plates (one control, and one doxycycline-treated). Inducer was added to the wells of one plate (treated) to a concentration of 2 μl and allowed to grow for one week. Production of IgG from each culture was determined using anti-IgG-horseradish peroxidase in a BioCoat kit (Becton Dickinson). The kit comprises an antibody capture assay in that the IgG in the cell culture media binds to anti-IgG coated on the plates. A 100 μl aliquot of IgG-containing media was added to each well for 1 h at 25° C. After washing with Tris-buffered saline, anti-IgG-horseradish peroxidase was added and incubated for 1 h at 25° C. After washing, 100 μl 3,3′,5,5′-tetramethylbenzidine substrate was added and incubated for 8 min at 25° C. Stop solution (50 μl of 1M phosphoric acid) was then added, and Absorbance at 450 nm measured in a plate reader within 30 min. The binding capacity of each well is 0.6 μg IgG, so samples from induced cell lines may need to be diluted. Concentrations are determined from a standard curve (0.05-5 μg/ml) of commercially available purified IgG (Sigma). The amount of antibody from cultures without inducer increased slightly, whereas antibody titers from cultures with doxycycline inducer increased dramatically (as much as 10-fold) over three weeks FIG. 3.

### EXAMPLE 2

### Premature Senescence Enhances Protein Secretion in CHO Cell Lines

The effects of inducing premature senescence in CHO cell lines was investigated, since they are widely used in commercial production of single chain antibodies and immunoglobulin fragments. CHO cells were also chosen because they have been demonstrated to undergo senescence. A secretable alkaline phosphatase recombinant expression construct (SEAP, Clontech) was stably introduced into CHO cells to monitor enhanced protein expression. An IRES-containing retroviral construct was used for delivery of the native TetR that was engineered to include a nuclear localization signal. The Pantropic system (Clontech) was used to deliver the retroviral DNA into the cells as described in Example 1. Cells containing TetR were selected as described in Example 1. Senescence-triggering fragments from Cip/Kip and INK4A proteins were introduced into CHO cells as described in Example 1. Senescence-competent CHO cells were selected in grown in neomycin containing Iscove's modified Eagle's medium as described in Example 1. Induction of premature senescence was measured by plating 50,000 cells into 6 well plates and the expression of SA-β-Gal in these cells was measured in cultures lacking (control) or containing (treated) inducer. Every 2 days for a term of 8 days, cells were fixed with gluteraldehyde/formaldehyde (2%/4%) solution. Fixed cells were washed with PBS, then incubated overnight at 37° C. with X-Gal substrate at pH 6.0 (Dimri, G. P., et al., 1995, Proc Natl Acad Sci USA,. 92: p. 9363-7). The number of senescent cells is determined by counting cells under the microscope (about 10 fields at 20× magnification) at time intervals following addition of media (control) or media containing inducer (treated). It was expected that a high percentage of cells >85% will display staining for SA-β-Gal (See FIG. 5). CHO cell lines were isolated by limiting dilution and tested individually for enhanced production of SEAP.

Expression of SEAP from control CHO cells was monitored as enhanced alkaline phosphatase activity from cell culture supernatants using p-nitrophenol phosphate as substrate and monitoring the absorbance at 420 nm over 5 minutes in a BioMek plate reader. Increased secretion of SEAP from CHO cells under RP Shift was determined over one week time in the presence (induced) or absence (control) of 2 mM. The amount of SEAP measured from cultures without inducer increased slightly over one week, whereas that from cultures with doxycycline inducer increased as much as 30-fold in just a few days. CHO cells harboring empty vector displayed SEAP activities similar to those of cells containing the senescence-triggering factors without being exposed to the inducer doxycycline.

It will be understood in the art that production of antibody-related proteins in a non-hematopoietic cell as demonstrated herein illustrates the generality of the claimed reagents and methods to increase heterologous protein production for any such heterologous protein in any cultured eukaryotic cell.

It should be understood that the foregoing disclosure emphasizes certain specific embodiments of the invention and that all modifications or alternatives equivalent thereto are within the spirit and scope of the invention as set forth in the appended claims.

We claim:

1. A cell line comprising an recombinant expression construct, wherein the recombinant expression construct comprises a nucleic acid encoding one or a plurality of Cy motifs or one or a plurality of Ankyrin repeat motifs and wherein the recombinant expression construct further comprises an inducible transcription regulation element comprising at least two tetracycline operator elements, wherein the cell line is a hydridoma fusion partner cell line.

2. The cell line of claim 1, wherein the cell line is a myeloma cell line or cell line suitable for fusion with mouse splenocytes to generate hybridoma

3. The cell line of claim 1, wherein the recombinant expression construct encodes one or a multiplicity of Cy motifs having an amino acid sequence (Lys/Arg)-Xaa-Leu, where Xaa is any amino acid.

4. The cell line of claim 2, wherein the recombinant expression construct encodes one or a multiplicity of Cy motifs having an amino acid sequence (Lys/Arg)-Xaa-Leu, where Xaa is any amino acid.

5. The cell line of claims 3 or 4, wherein the recombinant expression construct encodes a multiplicity of Cy motifs having an amino acid sequence (Lys/Arg)-Xaa-Leu, where Xaa is any amino acid, wherein the multiplicity of Cy motifs are expressed as a peptide multimer of said motifs.

6. The cell line of claims 5 wherein the Cy motifs encode a peptide having an amino acid sequence identified by SEQ ID Nos. 6 through 21.

7. The cell line of claims 6, wherein the recombinant expression construct encodes a multiplicity of Cy motifs having the same or different amino acid sequences.

8. The cell line of claim 1, wherein the recombinant expression construct encodes one or a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid.

9. The cell line of claim 2, wherein the recombinant expression construct encodes one or a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid.

10. The cell line of claims 8 or 9, wherein the recombinant expression construct encodes a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid, wherein the multiplicity of Ankyrin repeat motifs are expressed as a peptide multimer of said motifs.

11. A method of increasing yield of a protein from a eukaryotic cell culture, comprising:
(a) contacting a cell culture according to claim 1 with a compound that induces an inducible transcription regulation element comprising a tetracycline operator element; and
(b) collecting a protein fraction from the cell culture.

12. The cell line of claim 11, wherein the recombinant expression construct encodes one or a multiplicity of Cy motifs having an amino acid sequence (Lys/Arg)-Xaa-Leu, where Xaa is any amino acid.

13. The cell line of claim 11, wherein the recombinant expression construct encodes a multiplicity of Cy motifs having an amino acid sequence (Lys/Arg)-Xaa-Leu, where Xaa is any amino acid, wherein the multiplicity of Cy motifs are expressed as a peptide multimer of said motifs.

14. The cell line of claims 11 or 12, wherein the Cy motifs encode a peptide having an amino acid sequence identified by SEQ ID Nos. 6 through 21.

15. The cell line of claims 14, wherein the recombinant expression construct encodes a multiplicity of Cy motifs having the same or different amino acid sequences.

16. The cell line of claim 11, wherein the recombinant expression construct encodes one or a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid.

17. The cell line of claims 16, wherein the recombinant expression construct encodes a multiplicity of Ankyrin repeat motifs of a having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid, wherein the multiplicity of Ankyrin repeat motifs are expressed as a peptide multimer of said motifs.

18. An recombinant expression construct comprising: (a) a minimal promoter comprising a TATA sequence; (b) two phased operators downstream from the TATA sequence; and (c) two phased operators upstream of the TATA sequence.

19. The recombinant expression construct of claim 18, wherein the recombinant expression construct encodes one or a multiplicity of Cy motifs having an amino acid sequence (Lys/Arg)-Xaa-Leu, where Xaa is any amino acid.

20. The recombinant expression construct of claim 19, wherein the recombinant expression construct encodes a multiplicity of Cy motifs having an amino acid sequence (Lys/Arg)-Xaa-Leu, where Xaa is any amino acid, wherein the multiplicity of Cy motifs are expressed as a peptide multimer of said motifs.

21. The recombinant expression construct of claims 19 or 20, wherein the Cy motifs encode a peptide having an amino acid sequence identified by SEQ ID Nos. 6 through 21.

22. The cell line of claims 20, wherein the recombinant expression construct encodes a multiplicity of Cy motifs having the same or different amino acid sequences.

23. The recombinant expression construct of claim 18, wherein the recombinant expression construct encodes one or a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid.

24. The recombinant expression construct of claim 23, wherein the recombinant expression construct encodes a multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid, wherein the multiplicity of Ankyrin repeat motifs having an amino acid sequence Xaa-Xaa-Xaa-His-Asp-Ala-Ala-Arg-Xaa-Gly-Phe-Leu-Asp-Thr-Leu-Xaa-Xaa-Leu, where Xaa is any amino acid are expressed as a peptide multimer of said motifs.

25. The cell line of claim 1, wherein the hydridoma fusion partner cell line is Sp2/0-Ag14 Mouse B cell myeloma; YB2/0 Rat B lymphoblast; K6H6/B5 Human B lymphoma/Mouse myeloma; NS1 Human lymphoblast; FO Mouse myeloma; Y3-Ag 1.2.3 Rat myeloma; or P3×63-Ag8-653 Human myeloma.

26. The cell line of claim 1, wherein the cell line is a CHO cell line.

27. The cell line of claim 1, wherein the cell line is a NS0 cell line.

28. A method of producing a hydridoma cell comprising fusing a cell of the hydridoma fusion partner cell line for claim 1 with an antibody-producing murine spleen cell.

29. The method of claim 28, wherein the hydridoma cell produces an antibody.

30. The cell line of claims 8 or 9, wherein the ankyrin repeat motifs encode a peptide having an amino acid sequence identified by SEQ ID Nos. 22 through 25.

31. The cell line of claim 10, wherein the ankyrin repeat motifs encode a peptide having an amino acid sequence identified by SEQ ID Nos. 22 through 25.

32. The cell line of claims 16 or 17, wherein the ankyrin repeat motifs encode a peptide having an amino acid sequence identified by SEQ ID Nos. 22 through 25.

33. The cell line of claims 23 or 24, wherein the ankyrin repeat motifs encode a peptide having an amino acid sequence identified by SEQ ID Nos. 22 through 25.


SEQUENCE LISTING (25 sequences)

SEQ ID NO: 1 - protein, 40 aa, Homo sapiens
MSITKDQIIEAVAAMSVMDVVELISAMEEKFGVSAAAAVA

SEQ ID NO: 2 - DNA, 23 bp, Homo sapiens
aggatgttataaagcatgagtca

SEQ ID NO: 3 - DNA, 23 bp, Homo sapiens
ggagggcatggatgctaagtcac

SEQ ID NO: 4 - DNA, 26 bp, Homo sapiens
tctcccttctccaaccggaggatcac

SEQ ID NO: 5 - protein, 18 aa, Homo sapiens
  misc_feature (1)...(3): Xaa stands for any amino acid
XXXHDAARXGFLDTLXXL

SEQ ID NO: 6 - protein, 6 aa, Homo sapiens
KRRLDL

SEQ ID NO: 7 - protein, 6 aa, Homo sapiens
KRKLDL

SEQ ID NO: 8 - protein, 6 aa, Homo sapiens
KRRLEL

SEQ ID NO: 9 - protein, 6 aa, Homo sapiens
KRRLFG

SEQ ID NO: 10 - protein, 6 aa, Homo sapiens
KRRLFV

SEQ ID NO: 11 - protein, 6 aa, Homo sapiens
GRRLVF

SEQ ID NO: 12 - protein, 6 aa, Homo sapiens
PRNLLS

SEQ ID NO: 13 - protein, 6 aa, Homo sapiens
PRRLLF

SEQ ID NO: 14 - protein, 6 aa, Homo sapiens
CRSLFG

SEQ ID NO: 15 - protein, 6 aa, Homo sapiens
CRNLFG

SEQ ID NO: 16 - protein, 6 aa, Homo sapiens
CRRLFG

SEQ ID NO: 17 - protein, 6 aa, Homo sapiens
KRRLIF

SEQ ID NO: 18 - protein, 6 aa, Homo sapiens
KRRLFT

SEQ ID NO: 19 - protein, 6 aa, Homo sapiens
LKKLFS

SEQ ID NO: 20 - protein, 6 aa, Homo sapiens
LKKLSG

SEQ ID NO: 21 - protein, 6 aa, Homo sapiens
KRSLKL

SEQ ID NO: 22 - protein, 21 aa, Homo sapiens
RPVHDAAREGFLDTLVVLHRA

SEQ ID NO: 23 - protein, 21 aa, Homo sapiens
SPVHDAARTGFLDTLKVLVEH

SEQ ID NO: 24 - protein, 21 aa, Homo sapiens
AVIHDAARAGFLDTLQTLLEF

SEQ ID NO: 25 - protein, 21 aa, Homo sapiens
RPVHDAAREGFLDTLVVLHRA